(19) United States
(12) Patent Application Publication
(10) Pub. No.: US 2008/0039905 A1
(43) Pub. Date: Feb. 14, 2008

(54) Electronic Device and Its Usage in the Bio-Resonance Functional Medicine

(76) Inventors: Ion Tomescu, Bucharest (RO); Emilian Dobrea, Giurgiu (RO)

Correspondence Address: HERSHKOVITZ & ASSOCIATES, 2845 DUKE STREET, ALEXANDRIA, VA, 22314, US

(21) Appl. No.: 11/663,297

(22) PCT Filed: Sep. 15, 2005
(86) PCT No.: PCT/RO05/00012

§ 371 Date: Mar. 21, 2007

(30) Foreign Application Priority Data
Sep. 21, 2004 (RO) A2004 00806
Aug. 17, 2005 (RO) A2005 00718

(51) Int. Cl. A61N 1/02

(52) U.S. Cl. 607/66

(57) ABSTRACT

This invention relates to an electronic device and the use thereof in the bio-resonance functional medicine by generating electric signals with positive offset, sinus, square or triangle wave shapes, selectable frequency between a lower limit and an upper limit, with variable gain numerically controlled by a microprocessor that can provide a constant current or a modulated current through an external circuit, that may be inside a human body, with selectable on/off times and may provide simultaneous generation of two independently programmable and electric isolated electric signals, that may be applied in two circuits inside the human body and that is able to generate procedures, functions or treatments wherein a procedure generates the signal type having the corresponding parameters, a function comprises a sequence of procedures and a treatment comprises a sequence of functions and procedures performed in order to detect the presence of a microorganism inside the human body and to eliminate this microorganism from the human body respectively, using its own database or the professional support of the specialized medical personnel, characterized by generating and transmitting to the patient of a procedure, function or treatment personalized according to telemedicine standards requirements.

This invention describes a device and its usage in the bio-resonance functional medicine.

Special designed equipments, built to generate resonant frequencies and wave shapes intended to eliminate parasites, bacteria, fungi or viruses and also to charge with energy the glands and organs of the body, are known in the art. Some of these equipments are able to automatically sweep two selected frequency limits through the body and to display the list of parasites, bacteria, fungi and viruses found in the body.

Other equipments are known, that are designed and built to individually generate the amplitude and frequency that destroy the selected parasite, fungus or virus, the next sweep determining if the said parasite/bacteria/fungus/virus is still present or was eliminated, where this procedure is applied for each component of the detection list.

Other simpler equipments are also known, named ZAPPER, that generate square signals having a frequency between 2,000 and 40,000 Hz and a period of application of 7 minutes, followed by a 20 minutes pause, this cycle being repeated three times.

The most equipments manufactured by now are designed following the researches published by Dr. Hulda Clark, in her books like the “Cure of All Diseases”.

The drawbacks of these equipments are:
the ZAPPER type equipments are designed to be manually used by each individual person, who is informed about the device characteristics and operation; this person is not able to generate complex functions required by a particular type of treatment, recommended by the specialized literature or medical staff, or series of treatments that are specific for each particular case;
other types of equipment may be used only in special medical practices, with very high expenses, which make them accessible only to very wealthy people;
for specific treatments using this type of equipments, it is important to note that the procedures require a large amount of information regarding the patient, that may not be objectively acquired;
the patient doesn't have his own control procedures regarding the efficiency of the treatment.

The technical issue this invention address is to make available a device and a method to use this device, allowing to automatically generate a sequence of functions (complex procedures) corresponding to single or combined treatments and to achieve complex procedures that may be similar or different.

The device according to this invention comprises two independent identical channels, electrically isolated to each other, one channel containing a DDS (Direct Digital Synthesizer) generator module that can output electric signals with sine, square or triangle shape and variable frequency according to the commands received from a microprocessor, a preamplifier circuit that drives the signal from the DDS module to the final amplifier, whose gain is digitally controlled by the same microprocessor, a switching module for the output circuit, providing the direct or reverse coupling of the output signal, amplified by the final amplifier, to one of the external circuits selected by the microprocessor commands, a circuit that galvanic isolates the command cables USB1 and USB2, making possible this way to use a single PC/laptop to command the two independent channels and still maintain the galvanic isolation between the said channels, a stabilized power supply module that provides separate, galvanic isolated, electric supplies for the two channels, and four connecting circuits between the device and the external circuits, coupling the signal generated to one of the external circuits, that may consist, for a human body, of right hand—left hand or right hand—left foot or right hand—right foot for the channel 1 and left hand—right foot or left hand—left foot or right foot—left foot for the channel 2, a microprocessor that generates, using special designed software, commands for the generation of electrical signals having shapes and frequencies within the accepted boundaries of the DDS frequency synthesizer module, together with gain selection commands for the signal amplifier and command for the reverse coupling of the amplified signal to the external circuit, commands for reading the current intensity in this circuit, setting commands for the digital/analog converter that, together with a differential electronic amplifier, outputs a pre-selected voltage that allows the reading of the output current values around this pre-selected value, the same microprocessor being connected by an USB link to a personal computer or a laptop that manages a database and runs software to generate procedures, functions or treatments.

The use of the device according to this invention allows to program and to automatically generate a procedure, a function (complex procedure) or treatment respectively, wherein a procedure consists of selecting the wave shape, the frequency, the sweeping range of frequency, the step and the rate of frequency variation, the digital value of final gain, the ON and OFF times, the total coupling time, the selected external circuit, the positive and negative (percentage) adjusting limits for the selected gain during the coupling period, direct coupling or reverse coupling of the connections to this circuit and so on, and wherein a function (complex procedure) represents a sequence of procedures, the number of repetitions for each procedure and for reading the current value in the output circuit and wherein a sequence of functions and procedures is employed to generate a treatment that includes elimination of the selected parasites, bacteria, fungi or viruses, followed by the energetic charge of the human body organs affected by the presence of the parasites, bacteria, fungi or viruses and an elimination checking procedure, that is a function for resonance detection of their presence inside the human body.

The intention of the device and its method of use is to give the necessary information to the user to decide if it is necessary to ask specialized assistance of a physician for his/her diet quality or for the presence in his/her body of microorganisms detected by the device and then to use the advises of the qualified experts, given as automatic treatments, or to directly consult specialized physicians. The communication between the user and specialized medical staff relies on the long distance communications using the Internet infrastructure, the user/patient being able to automatically send to a physician/hospital the diagrams he/she has acquired by executing the personal resonance tests, and the physician/expert may build a personalized treatment that may be sent back using an export procedure to the patient over the Internet. The patient can import this treatment in his/her database and automatically apply it, thereafter repeating the personal tests and, if necessary, the remote dialogue with the selected physician.

It is important to note that using the resonant process provided by the device according to this invention one can determine the presence or absence of the microorganisms along the selected circuit, that may include one of the human body circuits as e.g. the following circuits: Right Hand—Left Hand, or Right Hand—Left Foot, or Right Hand—Right Foot, or Left Hand—Right Foot, or Left Hand—Left Foot, or Right Foot—Left Foot.

After accomplishing the detection in one of the aforesaid circuits, the procedure of elimination of the microorganism whose presence was detected inside the human body may be selected, in an order brought by a person skilled in the art, the existence of the two electric isolated circuits, that can simultaneous accomplish complex procedures, bringing a real benefit for the patient.

The checking of microorganism removal is acquired by using the AUTOTEST procedure that allows to detect the presence or absence of that particular type of microorganism along the selected external circuit, both if the circuit belongs to the human body or it is a food testing circuit or something equivalent.

This invention has the following advantages:
the device and method according to this invention allow to automatically generate a sequence of functions (complex procedures) corresponding to single or combined treatments, prescribed by the technical literature or by the specialized medical staff. Furthermore, the simultaneous or separate usage of two identical, electrically isolated to each other, independently programmable channels, allows to execute complex procedures, identical or different, on external circuits applied to the same patient or to two different patients, or on test circuits corresponding to various types of alimentary or technical tests;
the procedure and method according to this invention may be used within industrial or alimentary procedures of detection by resonance of various metals, substances, microorganisms in the food, water and so on, detection of electromagnetic resonance phenomena inside electric or electronic circuits or detection of various microorganisms inside the animal or human body and, if necessary, may be used to destroy those microorganisms, using procedures recommended by the persons skilled in the art;
in other technical fields, this invention may act as a function generator specialized for other industrial applications.

The following is an embodiment of this invention, that is described in connection with FIGS. 1 to 19, that represent:

FIG. 1, a block diagram of the electronic device;

FIG. 2, the schematic of the power supply module;

FIG. 3, the block diagram of one of the electronic signal generator module's channels;

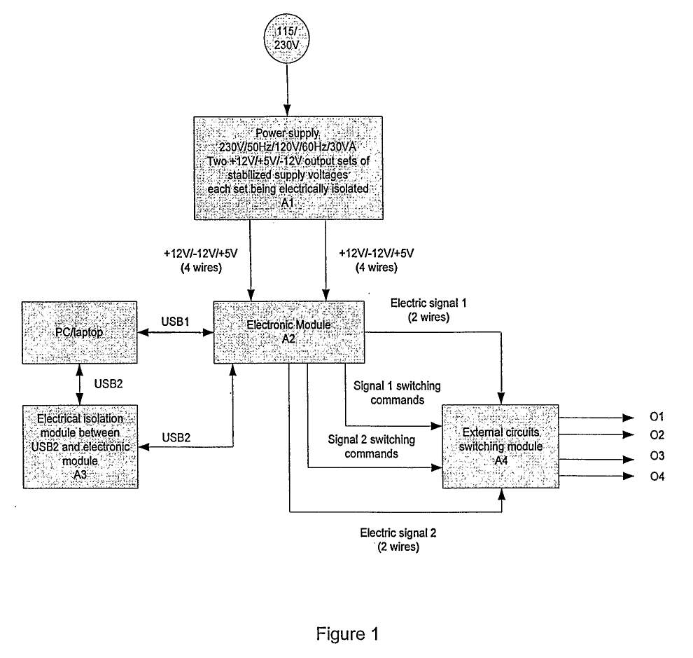

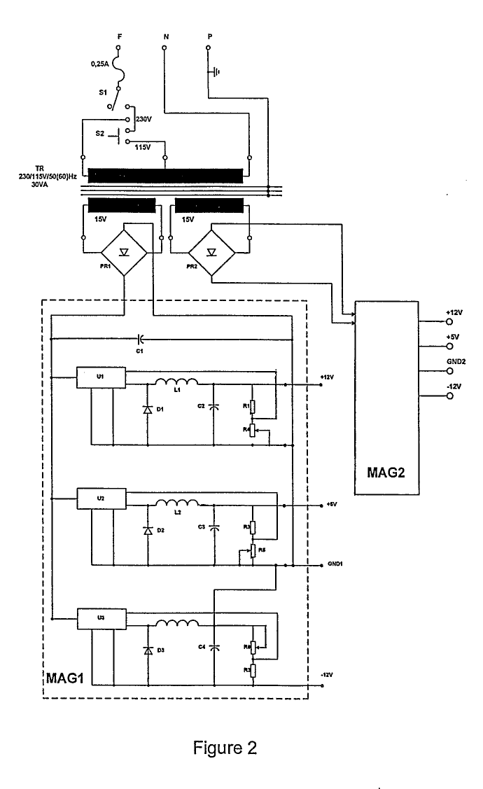

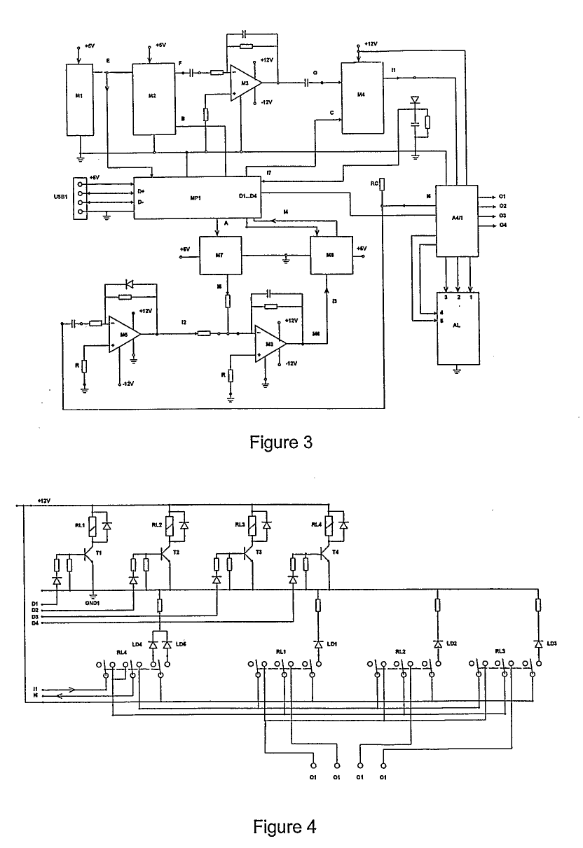

FIG. 4, the switching module schematic;

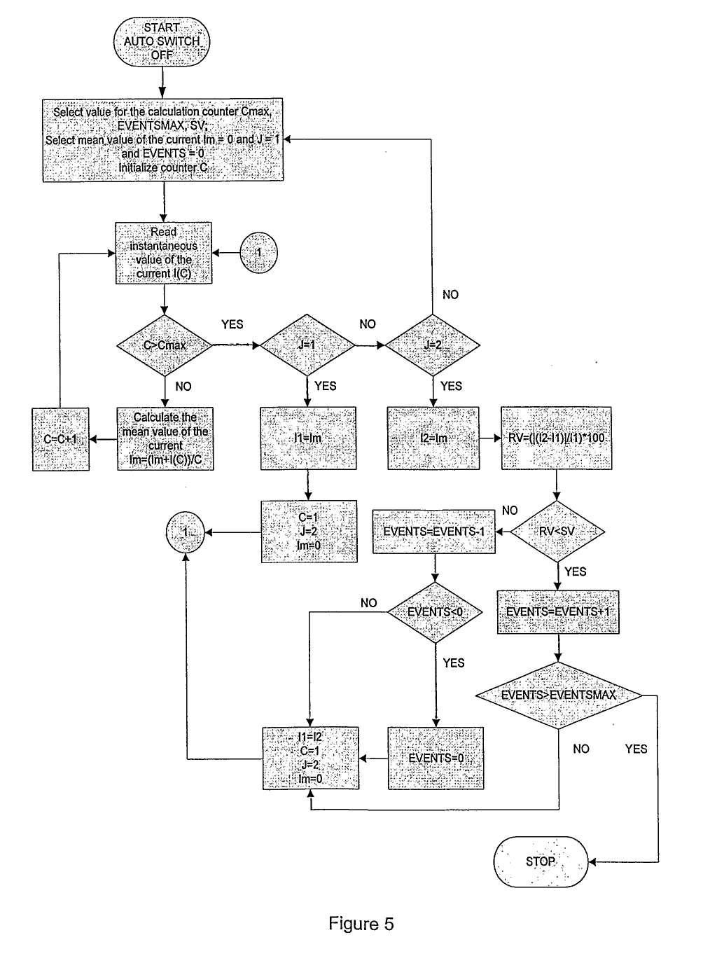

FIG. 5, method for determining the current value gradient in the selected external circuit (channel 1 or 2);

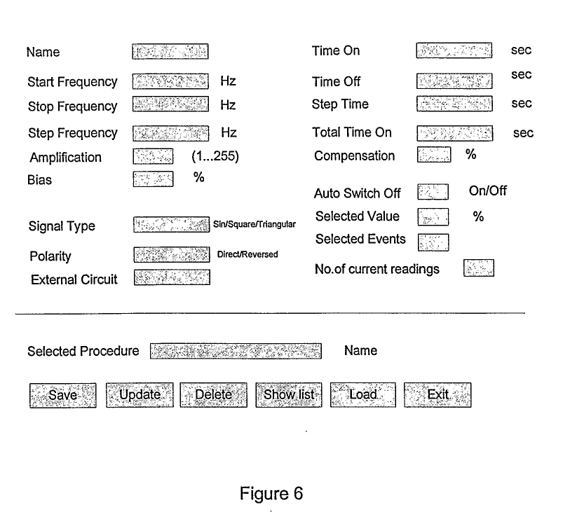

FIG. 6, graphical interface for procedure programming;

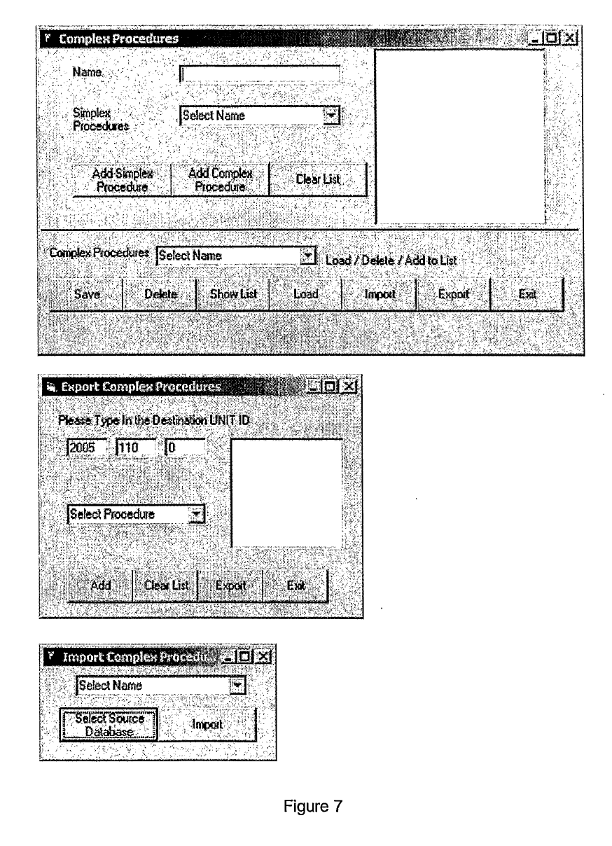

FIG. 7, graphical interface for function programming;

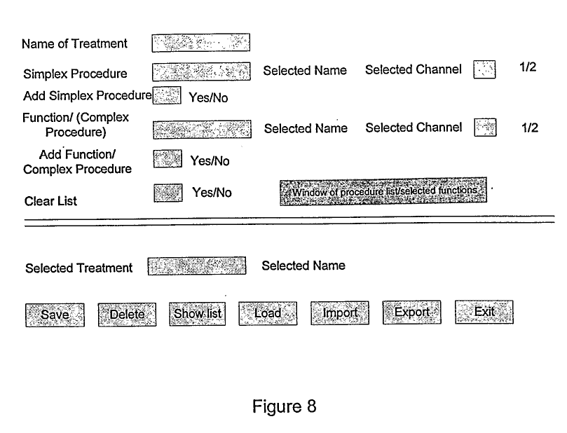

FIG. 8, graphical interface for treatment programming;

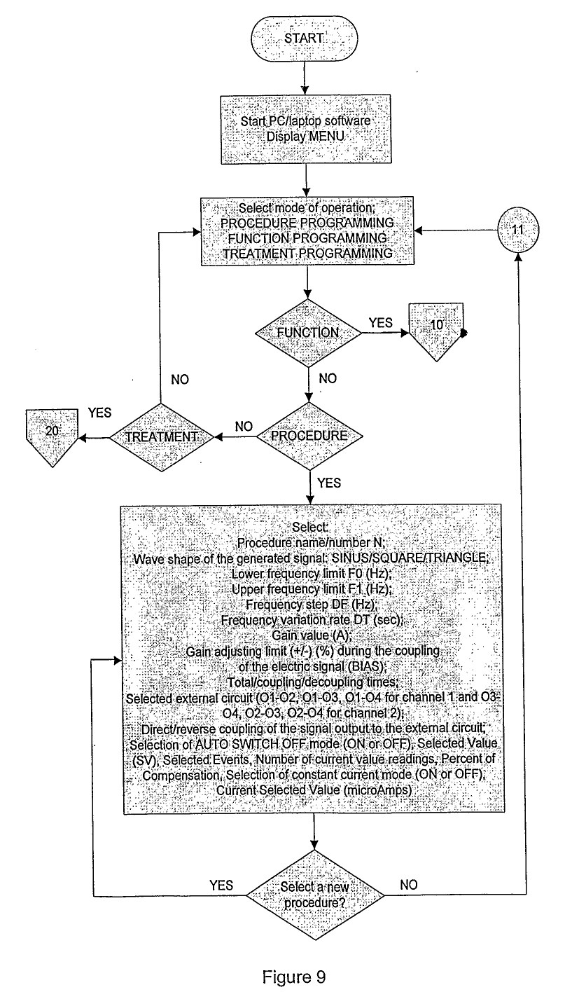

FIG. 9, method for procedure programming;

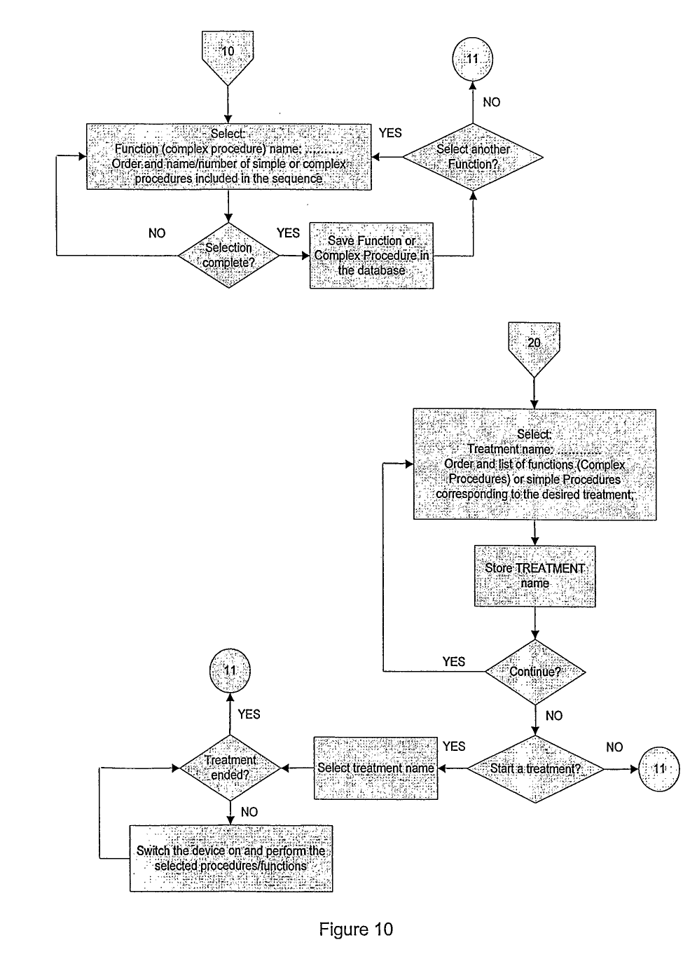

FIG. 10, method for function (complex procedure) and treatment programming;

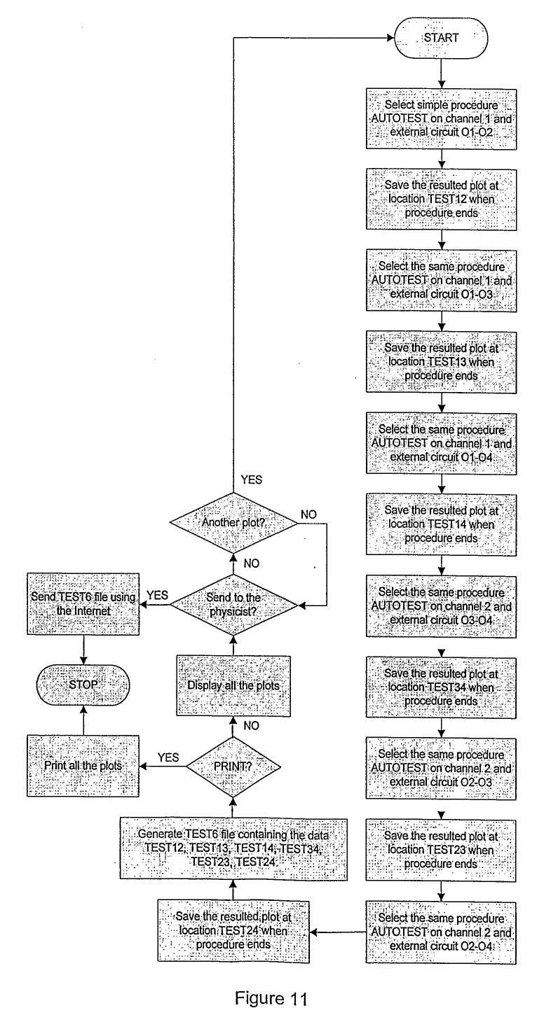

FIG. 11, programming method for a function that automatically and sequentially measures the frequency response in the external circuits O1-O2 and O1-O3 and O1-O4 for channel 1 and O3-O4 and O2-O3 and O2-O4 for channel 2;

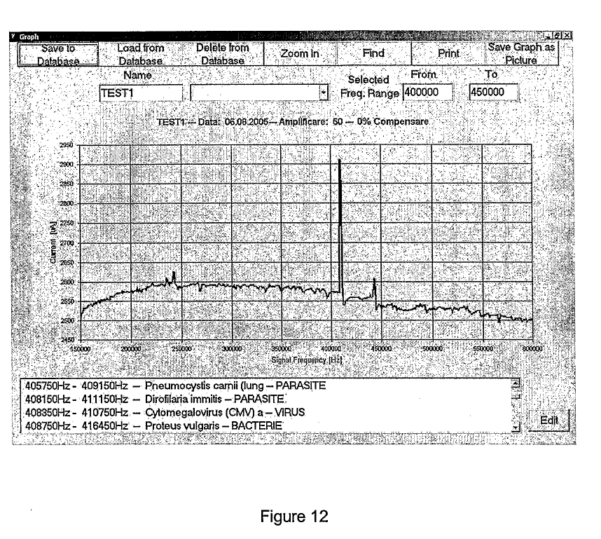

FIG. 12, graphical example of real frequency response in one of the selected external channels, namely right hand—left hand of a human body having excessive quantities of microorganisms along the channel 1;

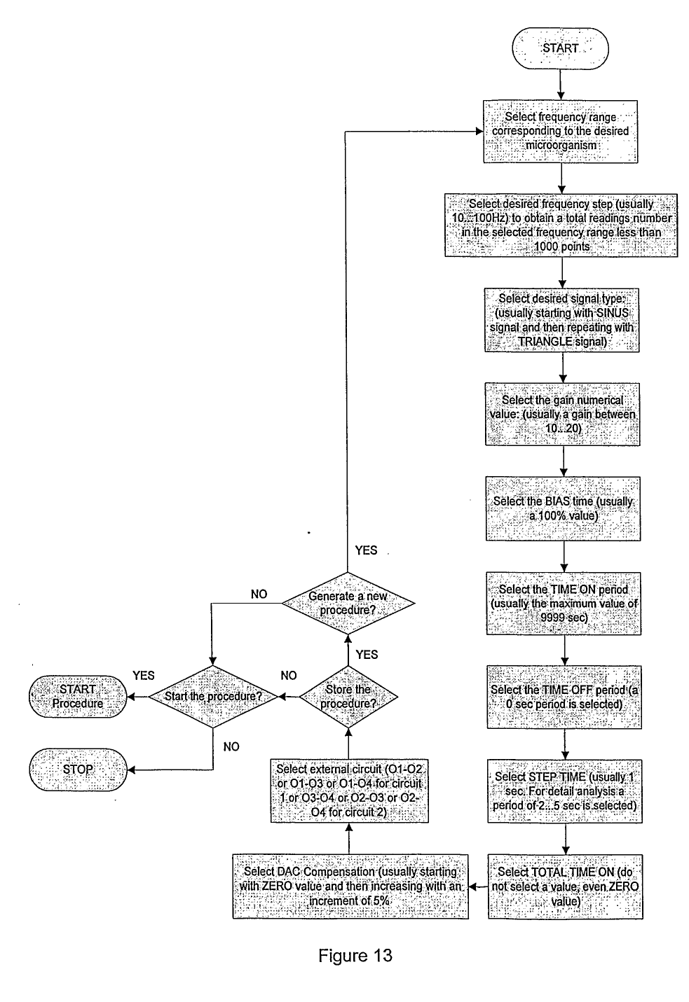

FIG. 13, programming method for a function that measures the frequency response of a parasite or virus or fungus/mildew in one of the selected external circuits (channel 1 or 2);

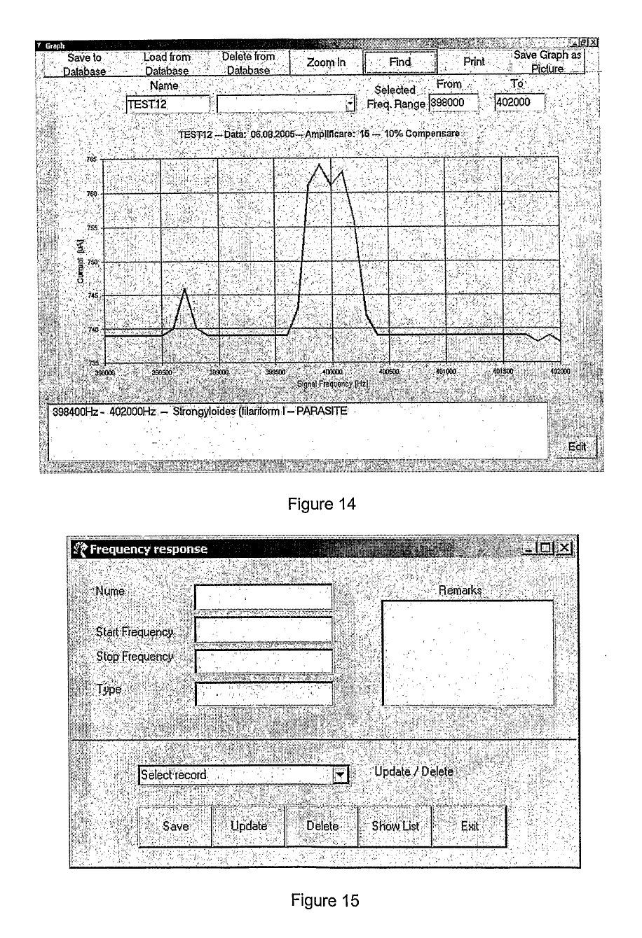

FIG. 14, graphical example of an actual frequency response of the external circuit right hand—left hand of a human body infested with the parasite Strongyloides in the channel 1;

FIG. 15, graphical interface for completing the database of microorganisms and their resonance frequencies;

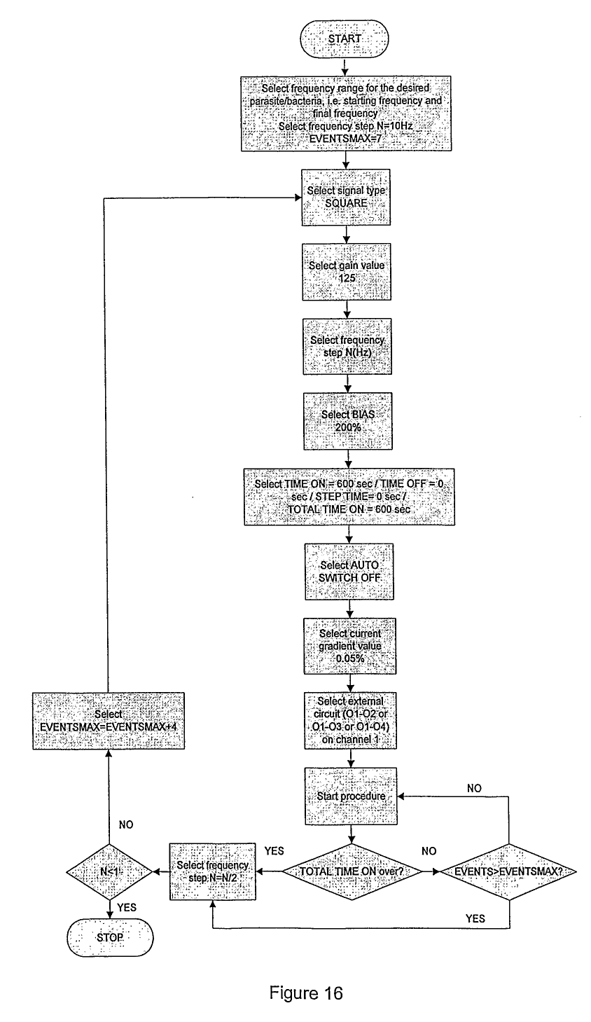

FIG. 16, method of treatment for function (complex procedure) programming to eliminate a parasite/bacteria;

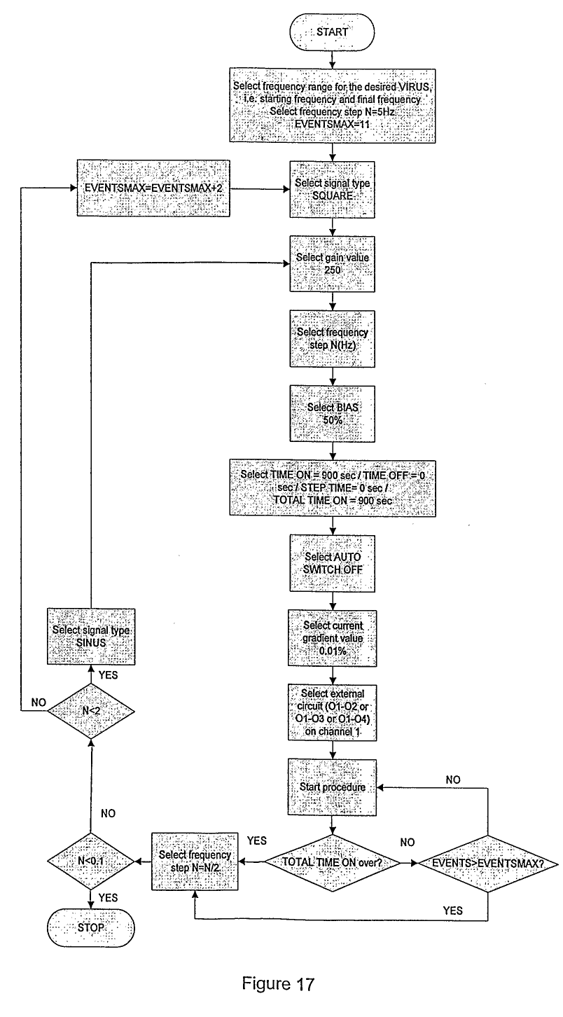

FIG. 17, method of treatment for function (complex procedure) programming to eliminate a virus;

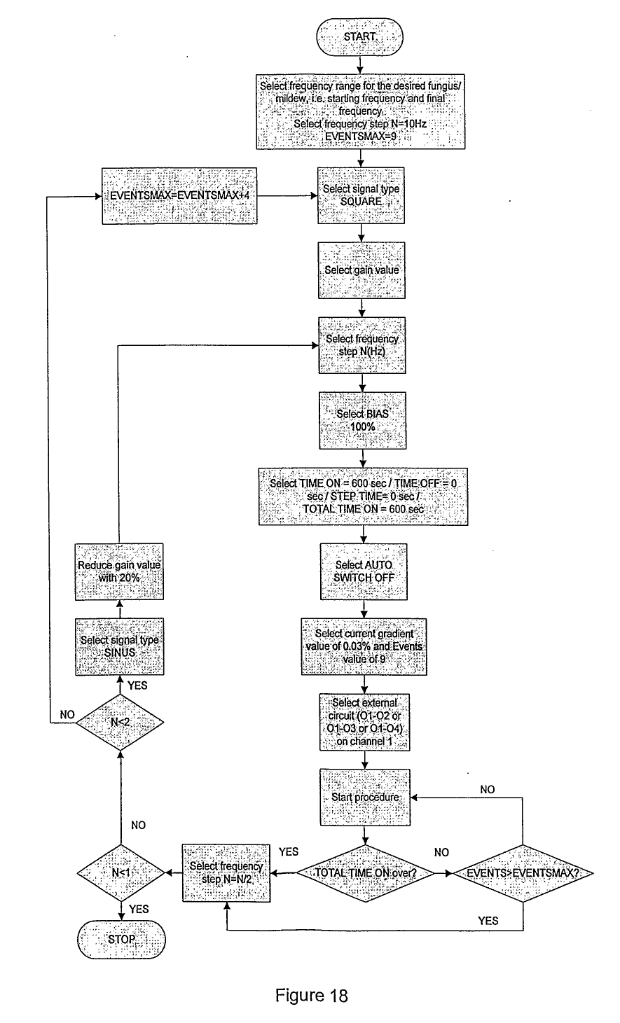

FIG. 18, method of treatment for function (complex procedure) programming to eliminate a fungus/mildew;

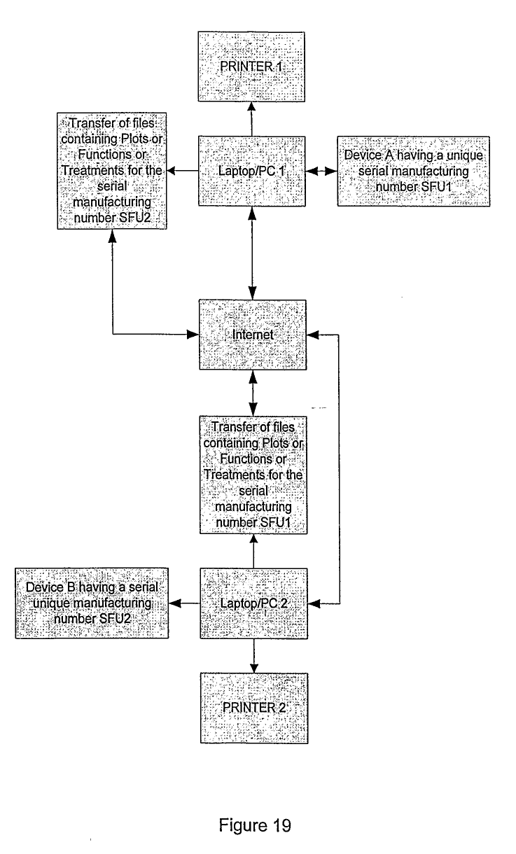

FIG. 19, communication/usage in the telemedicine network-block diagram.

The device and its method of use according to this invention generate electric signals whose wave shape, amplitude, frequency, modulation and duration can be programmed. These signals may be generated as single complex functions or as a sequence of functions having the same complexity, imposed by a predetermined treatment or recommended by medical personnel, having the qualification required by the standards of the functional medicine.

The electronic device generates the signals, measures the value of the current generated in the external circuit and displays the programmed parameters and the real-time measured parameters, according to the applied settings.

By using the device according to this invention, complex functions can be automatically generated, that command the electronic device to generate wave shapes, amplitudes, frequencies, frequency, period and amplitude modulations that are specific for a particular treatment designated to eliminate a particular parasite, bacteria, fungus or virus, localized on a specific organ, followed by specific procedures for energetic charge of the organs and glands affected by the presence of the said parasites, bacteria, fungi or viruses, or to generate a sequence of complex functions, that enable the body to eliminate a range of parasites (larvae and eggs). Bacteria, fungi and viruses in a list recommended by specialized medical personnel or detected by the electronic device using the resonance method.

The block diagram of the device according to this invention is shown in FIG. 1, wherein A1 is a power supply module, receiving 230 V/50 Hz or 120 V/60 Hz through a power supply switch and supplying two identical output voltage sets, isolated one from the other, of +12 V/−12 V/+5 V with a total power of 30 VA, that independently feed two identical channels belonging to electronic module A2. The electronic module comprises two identical channels, isolated one from the other, where each channel is an electric signal generator that outputs a sinus, square or triangle shape electric signal, having a frequency between 1 Hz and 10 MHz, with a minimum increment of 0.1 Hz, that is pre-amplified and feed into a power amplifier having a digitally controlled gain, able to output an electric signal with a maximum selectable amplitude of 12 Vpp (peak to peak), that is then direct or reverse coupled to one of the pairs O1-O2 or O1-O3 or O1-O4 for channel 1 and O2-O3 or O2-O4 or O3-O4 for the channel 2 respectively, selected by module A4. A 500 ohms resistor is provided in the output circuit of these channels that measures the electric current in the output circuit, having a maximum value of 24 mApp (peak to peak) when the outputs are short-circuited. All the components used must operate without limitations in the frequency domain from 1 Hz to 10 MHz. The electronic module A2 is controlled from the same PC/laptop using two cables USB1 and USB2, which would create a galvanic connection in the common ground and +5V power supply an subsequently a direct coupling between the two circuits, assumed to be built mutually isolated. This is the reason to use an isolating module between the USB2 output and the electronic module, namely A3, that is an USB Insulator known in the art, providing the electric isolation of USB2 communication with PC/laptop from the electronic module A2.

The two electric signals, independently generated and mutually isolated, are directed by the switching module A4 in six external circuits provided inside the human body using the connections O1 to O4, externally connected to the right hand, left hand, right foot and left foot, i.e. O1-O2, O1-O3, O1-O4, O2-O3, O2-O4 and O3-O4, each of which being able to be direct or reverse coupled.

The same circuits may be used to test electronic equipments or laboratory samples. According to FIG. 2, each channel is supplied with DC stabilized voltages of +12 V/−12 V/+5 V, independently generated by the two secondary 15 V coils of the power transformer TR, with a rectifier bridge PR and three power switching regulators U1, U2 and U3 of LM2575 type, that output +12 Vcc, −12 Vcc and +5 Vcc voltages at a maximum current of 1 A, that form the power supply module MAG1 for the channel 1 and an identical, but electrically isolated, module MAG2 for the channel 2.

The electrolytic capacitors C1 have a value of 1000 microfarads at a nominal voltage of 50 Vcc, and the electrolytic capacitors C2, C3 and C4 are identical and have a value of 100 microfarads at a nominal voltage of 15 Vcc.

The inductances L1, L2 and L3 are identical, 220 microhenri/1 A.

The Schottky diodes D1, D2 and D3 have nominal values of 1 A/25 Vcc. The resistors R1, R2 and R3 are identical, with a value of 10 Kohms. The resistors R4, R5 and R6 are variable resistors, with a value of 25 Kohms. The function of these switching regulators is known in the art and described in the literature.

FIG. 3 shows the block diagram of the A2 electronic module's channel 1, wherein a circuit M1, a fixed frequency quartz stabilized signal generator, generates at the output E the reference signal of e.g. 24 MHz, that goes to a digital frequency synthesis circuit M2, for example a DDS circuit AD9833 manufactured by Analog Devices, USA, that supply at the output F a sinus, square or triangle signal, with variable frequency, e.g. from 1 Hz to 10 MHz, with a minimum increment of 0.1 Hz, controlled by the programming values sent by the microprocessor MP1 to the B input; the said signal is applied to a preamplifier integrated circuit M3, that is able to operate normally in this frequency range and that is connected at the output G with the input of the integrated final amplifier M4, the gain of which is digitally programmed by the microprocessor MP1 at the input C. The output I1 of the amplifier M4 is closed through the external circuit O1-O2 or O1-O3 or O1-O4 and through the resistor RC, having a value of 500 ohms, the voltage across the resistor RC (directly proportional with the current that flows through RC) being applied to an amplifier/rectifier circuit M5 that outputs at the output I2 a DC voltage proportional with the value of the current (mA) generated by the output circuit. The circuit M6 is differential/integrator amplifier that supplies at the output I3 a DC voltage proportional with the mean value of the difference between the DC voltage generated by the digital/analog converter M7 at the output I5 and the output current, this difference being read by the analog/digital converter M8 and sent to the microprocessor MP1 at the input 14.

The microprocessor MP1 provides to the outputs A, B, C the programming commands for the circuits M2, M4 and M7, and to the outputs D1 . . . D4 the switching commands for the module A4/1. The LED display AL receives at the inputs AL1 . . . AL3 the ON command for the LED's situated on the panel of the electronic module A2, corresponding to the external circuit selection O1-O2 or O1-O3 or O1-O4 and at the inputs AL4 . . . AL5 the ON command corresponding to the DIRECT/REVERSE selection. The channel 2 of the same module A2 is built identically.

The device according to this invention allows to generate a fixed frequency, with a sinus, square or triangle wave shape, or a sweep between two frequency limits, with programmable wave shape and values, with programmable step and duration, signals that are pre-amplified with a fixed gain by the circuit M2 and amplified by the integrated circuit M4 whose gain is digitally controlled and that can automatically maintain preprogrammed constant values for the currents at the outputs O1 . . . O4 or can provide an amplitude modulation of the output current with a preprogrammed function. Furthermore, the device may provide a constant value for the gain of the circuit M4 while varying the frequency between programmable limits and measures the output current variations to determine the possible resonances in the circuit O1 . . . O4, values that are stored in the PC/laptop memory, and displays a Current (mA) vs. Frequency (Hz) plot or Impedance vs. Frequency plot.

The differential and integrator amplifier M6 together with the digital/analog converter M7 and the microprocessor MP1 provide the detection of resonant conditions by determining the mean current value for the first few hundred readings of the instantaneous current value at the beginning of the selected frequency range, by selecting a compensation level (by subtraction) for this mean value (from 0 to 100%) and by commanding the circuit M7 to generate this compensation voltage to the output I5. Subsequently, the mean current values (compensated or not), computed for selectable periods of time (in seconds), are stored together with the frequency generated, constituting the database used to display the Current (microampers) vs. Frequency (Hz) plot or Impedance vs. Frequency plot. The signal I7, proportional with the mean value of voltage at the output I1 of the final amplifier M4 is sent from the output I1 of the final amplifier M4 through a separator and integrator circuit to a analog/digital converter input of the microprocessor MP1.

FIG. 4 shows the schematic diagram of the switching module A4/1 corresponding to the channel 1, wherein the relays RL1, RL2, RL3, RL4 are switched by the transistors T1 . . . T4 commanded by the microprocessor MP1 (channel 1) of the module A2.

The electric signal generated in the channel 1 of the module A2 is coupled by the relay contacts of RL1, RL2, RL3 to one of the external circuits O1-O2 or O1-O3 or O1-O4 and the LEDs LD1 . . . LD3 optically display the selected circuits. The direct or reverse coupling of the electric signal generated in the channel 1 is provided by the relay RL4, its state being displayed by the LED's LD4 and LD5. Similarly, for channel 2 the connections with the circuits O2-O3 or O2-O4 or O3-O4 are provided by the relay contacts corresponding to channel 2 with the optical display being provided by the LED's corresponding to channel 2 and the direct or reverse coupling is provided by a relay corresponding to channel 2, its state being displayed by two LED's corresponding to the same channel 2.

FIGS. 9 and 10 shows the logical diagrams for programming procedures, functions and treatments.

FIG. 5 shows the logical diagram for the automatic stop option for a procedure (AUTO SWITCH OFF) when the variation gradient of the current in the selected external circuit reaches an inferior limit that defines the acquiring of a constant current in the said circuit. The real gradient value is designate as RV, and the calculations are based on averaged values of the instantaneous current measured in a number of readings Cmax (programmable parameter) and the selected value is designated as SV. The formula used to calculate the gradient is

$$RV = \frac{\vert I 2 - I 1 \vert}{I 1} * 100 ( \% )$$

The logical diagram uses the counter C, the value of which is incremented in steps of 1 to Cmax, the step being generated by the frequency changing rate during the procedure. The measured mean value of the current is designated as Im and the programming variable J may take the value 1 or 2, depending to the reading sequence, so that the value of current I2 is read after the value of current I1. When RV<SV, an event designated as EVENTS occurs and the condition of automatic stop of the procedure is activated when the number of event EVENTS exceeds the imposed maximum value EVENTSMAX.

FIG. 6 shows the graphical interface for programming/modifying/reading a procedure, wherein a new procedure is generated starting with giving a name to that procedure and then introducing the frequency limits, frequency step, digital value for gain, coupling/decoupling/maintaining time in seconds for the selected frequency/total coupling time, following the selected compensation value and automatic stop of procedure option in conditions described hereinbefore. Subsequently, the sinus, square or triangle signal type is selected, that directly or reversely (depending on the Polarity selected) couples with the selected external circuit, that may be decoupled (Null) or O1-O2 or O1-O3 or O1-O4 for channel 1 and Null or O3-O4 or O2-O3 or O2-O4 for channel 2. After completing the selection for all desired parameters, the procedure is saved in the database under the selected name, by using the Save button in the lower part of the window.

If one needs to use a procedure that already exists in the database, the name of this procedure is selected with the button in the lower part of the window, after selection being possible to modify (Update) or delete (Delete) the procedure or to load (Load) the procedure for an individual run in the device according to this invention. To exit this menu, the button Exit is selected. The list of simple procedures that exist in the database is displayed using the button Show List.

FIG. 7 shows the menu of a Complex Procedure (Function) built as a sequence of (simple) procedures or as a sequence of complex procedures, already present in the database, where the names of the simple procedures are selected with the button Simplex Procedures and added in the list of the new complex procedure using the button Add Simplex Procedure. The name of the selected procedure is displayed in the right side window. Similarly, a complex procedure may be selected. If an error occurs, the Clear List button should be pressed. When the selection operation is complete, the complex procedure is saved using the Save button.

If one needs to use a complex procedure that exists in the database, the name of this procedure is selected, after selection being possible to delete or load the procedure in order to be used by the device according to this invention.

If a complex procedure or a number of complex procedures need to be exported to a recipient owing a device according to this invention, uniquely identified by its serial manufacturing number (in this example 2005-110-0), then the button Export is pressed and the complex procedures from the displayed list are sequentially selected and added using the Add button to the list displayed in the right side window. When the list to be exported is ready, the Export button is pressed. The list of exported procedures takes the form of a file that can be delivered using the Internet; after receiving this file, the recipient can import the complex procedures in his/her own database, using the presented menu. It is important to note that these imported procedures are functional only with the device according to this invention having the serial manufacturing number used during the export process.

FIG. 8 shows the graphical interface for programming a treatment comprising simple and complex procedures and that can simultaneously use both channels of the device. The operation of this interface is equivalent with that of the complex functions.

FIGS. 9 and 10 shows the logical diagrams for programming the procedures, functions and treatments.

FIG. 11 shows a model for automatically and sequentially determining the frequency response of all the six external circuits, storing each plot in a predetermined location. The presented model is equivalent with the generation of a treatment, the difference being that the plots are automatically and sequentially determined and stored in predetermined locations that can be exported using the Internet to any possible destination, and the recipient doesn't need to posses a device according to this invention.

FIG. 12 shows a real plot designated as TEST12 of the right hand—left hand circuit of a human body. The analysis of this plot reveals that at specific frequencies, for example between 400,000 Hz and 450,000 Hz, a quantity of microorganisms is present that adsorb a current much higher than the measured mean level.

For a detailed display the desired frequency range is selected within the displayed plot, for example between 400,000 Hz and 450,000 Hz, then the button “Zoom In” is pressed to display the current values only between the two limits.

FIG. 13 shows the logical diagram for generating a simple scanning procedure, in a selected external circuit, in a desired frequency range. It should be noted that for such scanning procedures a low level of amplification is used and the said scanning procedure is repeated using sinus and triangle signal. This type of procedure is called Autotest.

FIG. 14 shows the actual result of such a scanning procedure in the right hand—left hand circuit of a human body infested with the parasite Strongyloides, having a resonance frequency, presented in the book of Dr. Hulda Clark, “The Cure of All Diseases”, between 398,000 Hz and 402,000 Hz. The resulting plot exhibit a strong resonance response in this frequency range. The device according to this invention that was employed for this test stores in its database, corresponding to the plots, the list that was published in the book of Dr. Hulda Clark, containing the names of microorganisms, their type and the corresponding frequency range, the device according to this invention being able, by pressing the “Find” button, to display in the lower window the list of microorganisms having resonance frequencies between the frequency limits selected in the “From” and “To” fields. To complete the database corresponding to the plots the “Edit” button is pressed and then the guidelines presented in FIG. 15 are followed.

FIG. 15 shows the graphical interface for completing a database, employed in the procedure of resonance plots determination. The application of this procedure may be extended also for other fields, in which case the corresponding list will contain other names and frequency ranges specific for that application.

FIG. 16 shows the logical diagram for programming a complex procedure for removing a parasite or bacteria whose presence was detected in the previously scanned external circuit, using data from the literature, this procedure being successfully used by the authors on their own bodies.

Similarly, FIG. 17 and 18 shows the logical diagrams for programming a complex procedure for removing a virus present in the human body and of a fungus or yeast respectively, according to the scanning results and data in the literature. These procedures were also successfully used by the authors on their own bodies. It should be noted that after a treatment of this type the scanning were repeated, confirming the vanishing of the resonances of the type presented in FIG. 14.

FIG. 19 shows the block diagram for a potential use of the device and method according to this invention in the telemedicine network, being possible for two owners of devices according to this invention to remotely use both diagnostic and treatment procedures, where the recipient of a treatment is a patient and the expeditor is a physicist specialized in the functional bio-resonance medicine.

According to this block diagram, each user has a device according to this invention and a laptop and a printer, and also an Internet connection that allows the data exchange between patients and physicists, irrespective of their geographical location or their nationality, with real time benefits for the treatment process.

1-11. (canceled)

12. An electronic device for functional resonance comprising at least two independent channels, identical and isolated one from the other, wherein one channel comprises a generator module that can deliver electric signals having one of a sinus, square and triangle wave shape and a variable frequency and phase, depending on programming commands received from a microprocessor, a preamplifier circuit that takes the signals generated by the generator module and delivers these signals to a final amplifier circuit whose gain and ramp are digitally controlled by the microprocessor, a switching module of an output circuit providing one of direct and reverse coupling of an amplified output of the final circuit to one of external circuits selected by commands of the microprocessor, a galvanic isolation module located between at least two command cables that allows one of a single PC and laptop to command the two independent channels and still keeping the galvanic isolation between these channels, a power supply module providing separate and galvanic isolated stabilized supply voltages for the two channels and four connecting circuits between the device and external circuits that feed the signal generated by the final amplifier circuit to one of the external circuits, a microprocessor providing, by using special designed software, commands for generating positive-offset electric signals with wave shapes, at least one of increasing and decreasing frequencies and phase within limits accepted by a frequency synthesis module together with commands for one of a fix gain and a positive/negative ramp gain selection of the generated signal by the final amplifier circuit and commands for reversing coupling of this amplified signal to the external circuit, commands for duration of a selected generation/non-generation period, commands for reading output voltage of the signal and the current value in this circuit, commands for setting at least one of a digital and an analog converter providing a pre-selected voltage level allowing reading of output current/impedance values near the pre-selected value, such configuration defining a dedicated device for an automatic determination, through a complex procedure or a function, of the current-frequency or impedance-frequency response within any selected external circuit O1-O2 or O1-O3 or O1-O4, for channel 1, and O3-O4 or O2-O3 or O2-O4, for channel 2, connected to the device outputs, where such frequency response can be performed sequentially for each external circuit connected to each channel or simultaneously, two by two, for any frequency domain, type of signal and amplitude, any such frequency response being automatically memorized within a dedicated database existing on one of the PC and laptop, and where the microprocessor is connected through a connection to one of the PC and laptop in order to receive appropriate commands existing within one of preprogrammed simplex procedures, complex procedures and functions generated locally from a local database or imported from a selected transmitter based on pre-accepted request and to transmit to the one of the PC and laptop the measured values of the voltage and current existing within the selected external circuit under test, all such commands being synchronized in order to obtain the desired frequency response process.

13. The electronic device of claim 12, wherein the device is configured to provide programming and automatic generation of one of a simple procedure, a complex procedure and a function respectively, wherein a simplex procedure consists of automatically generating an electric signal with programmed parameters, the program parameters comprising one of a sinus, square and triangle wave shape, a frequency, a phase, a frequency variation step and a rate, a gain value, a gain ramp, ON and OFF times for the signal coupling to the external circuit, positive and negative percentage adjusting limits for the selected gain during the coupling period, one of a direct coupling and a reverse coupling of the output, the selection of the external circuit from those available, reading the voltage and current values in the output circuit, real time displaying of one of current/frequency values and impedance/frequency values and of one of a current/frequency and impedance/frequency plot, and wherein a function comprises a sequence of complex procedures selected to be automatically executed in the selected order and wherein a sequence of functions and procedures is employed to draw the plots for current or impedance vs. frequency in the selected external circuits or to generate a dedicated procedure that can include elimination of one of selected parasites, bacteria, fungi and viruses or other microorganisms within the selected external circuit.

14. The electronic device of claim 12, wherein during a frequency sweeping between two selected limits, a mean value of voltage and current respectively are measured in the external circuit, or their deviation relative to a pre-selected value, that is determined using instantaneous values measured during a selected period of time and for each value of generated frequency and a current/frequency or impedance/frequency plot is displayed and wherein presence of positive peaks is an indication for energy absorption, respectively of active presence of one or more microorganisms, and presence of negative peaks is an indication for presence of one or more inactive microorganisms or toxins.

15. The electronic device of claim 12, wherein the device is configured to provide an off-line programming of one of simplex procedures, complex procedures and functions on an external support that comprises one of a PC and a laptop, generating a common database reflecting personal experience that can be shared using a dedicated import-export mechanism, via Internet, between owners of devices or using technical literature or a history of current/impedance-frequency plots and results obtained from such an experience can be applied within a specific area, and in that it allows to transfer parameters corresponding to each procedure and function using an USB connection to a microprocessor that is able to store these parameters and generate on-line the programmed type of signal together with real time reading of generated frequency value and current in the selected external circuit this signal is applied to.

16. The electronic device of claim 12, further comprising a two input differential amplifier configured for scanning and generation of a current-frequency plot or impedance-frequency plot process, wherein first of inputs is proportional with instantaneous value of current generated in the selected external circuit and a second input receives a signal generated by a digital/analog converter controlled by the microprocessor and has a zero value when real value of the current is measured or can have a value up to 100% of mean value of measured current for an electric signal having a frequency at a beginning of a sweeping range, then measuring, during a sweeping process, variations of this current is around the value pre-selected by the microprocessor and generated by the DAC.

17. The electronic device of claim 12, wherein output of an independent galvanic isolated channel is one of programmed and manually selected to provide one of a direct and a reverse coupling to an output circuit that can be selected, at turn, by one of a program and a manual selection to any of possible combinations of terminals of the device, wherein selection commands are contained in programming fields of one of a simplex procedure, complex procedure and function and are automatically generated by the microprocessor.

18. The electronic device of claim 12, wherein the device is configured to use a reading for one of instantaneous values and averaged values over selectable periods of time for voltage, current and frequency, one of a current/frequency plot and impedance/frequency plot generated, that can be stored in a graphic database and can be one of displayed, printed and exported using the Internet for analysis, wherein the plot can be displayed in detail using a “ZOOM IN” command between selectable frequency limits within sweeping limits of a scanning process and can have added a list of microorganisms and their resonance frequencies within a selected frequency range, the list being stored in a database attached to a “Plot Graph” operation mode.

19. The electronic device of claim 12, wherein the device is configured for a procedure for removing one of a parasite and a bacteria and uses linearly increasing amplitudes, between an initial value and a final value, of signal applied to selected output circuit, during entire coupling period of time where such circuit can be used in-vitro testing.

20. The electronic device of claim 12, wherein the device is configured for a procedure for removing a virus and uses linearly decreasing amplitudes, between an initial value and a final value, of signal applied to selected output circuit, during entire coupling period of time where such circuit can be used in-vitro testing.

21. The electronic device of claim 12, wherein the device is configured for constant values of current and are used in a procedure of removing at least one of a fungus, mildew and toxin in selected output circuit, during entire coupling period of time where such circuit can be used in-vitro testing.

22. The electronic device of claim 12, wherein the device is configured for an automatic stop option used during simple procedures, that can be activated or not activated and that allows to stop a simple procedure or to jump to the next simple procedure within a complex procedure or function, wherein real value of current gradient in selected external circuit is calculated, in percentage, using difference between two consecutive values of current averaged over a pre-selected number of readings, divided by first current value, the result being multiplied with value of 100 and this value is compared with a pre-selected value commanding automatic stop of running procedure if a number of consecutive measurements of real gradient value is less than selected value, such situation being named as a positive event or a negative event if real gradient value is greater than a selected one, and will add such event to a events counter waiting for achieving a consecutive number of events greater than the selected one in order to stop the running procedure.

23. A method for determining functional resonance, comprising providing an electronic device comprising at least two independent channels, identical and isolated one from the other, wherein one channel comprises a generator module that can deliver electric signals having one of a sinus, square and triangle wave shape and a variable frequency and phase, depending on programming commands received from a microprocessor, a preamplifier circuit that takes the signals generated by the generator module and delivers these signals to a final amplifier circuit whose gain and ramp are digitally controlled by the microprocessor, a switching module of an output circuit providing one of direct and reverse coupling of an amplified output of the final circuit to one of external circuits selected by commands of the microprocessor, a galvanic isolation module located between at least two command cables that allows one of a single PC and laptop to command the two independent channels and still keeping the galvanic isolation between these channels, a power supply module providing separate and galvanic isolated stabilized supply voltages for the two channels and four connecting circuits between the device and external circuits that feed the signal generated by the final amplifier circuit to one of the external circuits, a microprocessor providing, by using special designed software, commands for generating positive-offset electric signals with wave shapes, at least one of increasing and decreasing frequencies and phase within limits accepted by a frequency synthesis module together with commands for one of a fix gain and a positive/negative ramp gain selection of the generated signal by the final amplifier circuit and commands for reversing coupling of this amplified signal to the external circuit, commands for duration of a selected generation/non-generation period, commands for reading output voltage of the signal and the current value in this circuit, commands for setting at least one of a digital and an analog converter providing a pre-selected voltage level allowing reading of output current/impedance values near the pre-selected value, such configuration defining a dedicated device for an automatic determination, through a complex procedure or a function, of the current-frequency or impedance-frequency response within any selected external circuit O1-O2 or O1-O3 or O1-O4, for channel 1, and O3-O4 or O2-O3 or O2-O4, for channel 2, connected to the device outputs, where such frequency response can be performed sequentially for each external circuit connected to each channel or simultaneously, two by two, for any frequency domain, type of signal and amplitude, any such frequency response being automatically memorized within a dedicated database existing on one of the PC and laptop, and where the microprocessor is connected through a connection to one of the PC and laptop in order to receive appropriate commands existing within one of preprogrammed simplex procedures, complex procedures and functions generated locally from a local database or imported from a selected transmitter based on pre-accepted request and to transmit to the one of the PC and laptop the measured values of the voltage and current existing within the selected external circuit under test, all such commands being synchronized in order to obtain the desired frequency response process.

24. The method of claim 23, further comprising providing programming and automatic generation of one of a simple procedure, a complex procedure and a function respectively, wherein a simplex procedure consists of automatically generating an electric signal with programmed parameters, the program parameters comprising one of a sinus, square and triangle wave shape, a frequency, a phase, a frequency variation step and a rate, a gain value, a gain ramp, ON and OFF times for the signal coupling to the external circuit, positive and negative percentage adjusting limits for the selected gain during the coupling period, one of a direct coupling and a reverse coupling of the output, the selection of the external circuit from those available, reading the voltage and current values in the output circuit, real time displaying of one of current/frequency values and impedance/frequency values and of one of a current/frequency and impedance/frequency plot, and wherein a function comprises a sequence of complex procedures selected to be automatically executed in the selected order and wherein a sequence of functions and procedures is employed to draw the plots for current or impedance vs. frequency in the selected external circuits or to generate a dedicated procedure that can include elimination of one of selected parasites, bacteria, fungi and viruses or other microorganisms within the selected external circuit.

25. The method of claim 23, wherein during frequency sweeping between two selected limits, a mean value of voltage and current respectively are measured in the external circuit, or their deviation relative to a pre-selected value, that is determined using instantaneous values measured during a selected period of time and for each value of generated frequency and a current/frequency or impedance/frequency plot is displayed and wherein presence of positive peaks is an indication for energy absorption, respectively of active presence of one or more microorganisms, and presence of negative peaks is an indication for presence of one or more inactive microorganisms or toxins.

26. The method of claim 23, further comprising providing an off-line programming of one of simplex procedures, complex procedures and functions on an external support that comprises one of a PC and a laptop, generating a common database reflecting personal experience that can be shared using a dedicated import-export mechanism, via Internet, between owners of devices or using technical literature or a history of current/impedance-frequency plots and results obtained from such an experience can be applied within a specific area, and in that it allows to transfer parameters corresponding to each procedure and function using an USB connection to a microprocessor that is able to store these parameters and generate on-line the programmed type of signal together with real time reading of generated frequency value and the current in selected external circuit this signal is applied to.

27. The method of claim 23, wherein a two input differential amplifier is used in scanning and generation of a current-frequency plot or impedance-frequency plot process, wherein a first of inputs is proportional with instantaneous value of current generated in the selected external circuit and a second input receives a signal generated by a digital/analog converter controlled by the microprocessor and has a zero value when real value of current is measured or may have a value up to 100% of mean value of measured current for an electric signal having a frequency at a beginning of a sweeping range, then measuring, during a sweeping process, variations of this current is around the value pre-selected by the microprocessor and generated by the DAC.

28. The method of claim 23, wherein output of an independent galvanic isolated channel is one of programmed and manually selected to provide one of a direct and a reverse coupling to an output circuit that can be selected, at turn, by one of a program and a manual selection to any of possible combinations of terminals of the device, wherein selection commands are contained in programming fields of one of a simplex procedure, complex procedure and function and are automatically generated by the microprocessor.

29. The method of claim 23, further comprising reading for one of instantaneous values and averaged values over selectable periods of time for voltage, current and frequency, one of a current/frequency plot and impedance/frequency plot generated, that can be stored in a graphic database and can be one of displayed, printed and exported using the Internet for analysis, wherein the plot can be displayed in detail using a “ZOOM IN” command between selectable frequency limits within sweeping limits of a scanning process and can have added a list of microorganisms and their resonance frequencies within a selected frequency range, the list being stored in a database attached to a “Plot Graph” operation mode.

30. The method of claim 23, wherein the device is configured for one of:
(a) removing one of a parasite and a bacteria and using linearly increasing amplitudes, between an initial value and a final value, of signal applied to selected output circuit, during entire coupling period of time where such circuit can be used in-vitro testing;
(b) removing a virus and using linearly decreasing amplitudes, between an initial value and a final value, of signal applied to selected output circuit, during entire coupling period of time where such circuit can be used in-vitro testing; and
(c) for constant values of current and for removing at least one of a fungus, mildew and toxin in the selected output circuit, during entire coupling period of time where such circuit can be used in-vitro testing.

31. The method of claim 23, wherein the device is configured for an automatic stop option and is used during simple procedures, that can be activated or not activated and that allows to stop a simple procedure or to jump to the next simple procedure within a complex procedure or function, wherein real value of current gradient in selected external circuit is calculated, in percentage, using difference between two consecutive values of current averaged over a pre-selected number of readings, divided by first current value, the result being multiplied with value of 100 and this value is compared with a pre-selected value commanding automatic stop of running procedure if a number of consecutive measurements of real gradient value is less than selected value, such situation being named as a positive event or a negative event if real gradient value is greater than a selected one, and will add such event to a events counter waiting for achieving a consecutive number of events greater than the selected one in order to stop the running procedure.
